(19) United States
(12) Patent Application Publication
(10) Pub. No.: US 2016/0089206 A1
(43) Pub. Date: Mar. 31, 2016

(54) UV LED INTEGRATED FLEAM

(71) Applicant: Seoul Viosys Co., Ltd., Ansan-si (KR)

(72) Inventors: Esther Lee, Siheung-si (KR); Sang-Cheol Shin, Suwon-si (KR)

(21) Appl. No.: 14/871,876

(22) Filed: Sep. 30, 2015

(30) Foreign Application Priority Data
Sep. 30, 2014 (KR) 10-2014-0131981

(51) Int. Cl. A61B 19/02; A61L 2/10; A61B 17/3205

(52) U.S. Cl. CPC A61B 19/0262; A61B 17/3205; A61L 2/10; A61B 2217/002

(57) ABSTRACT

Provided is a UV LED-integrated fleam which can be used while sterilizing a needle. The fleam may include: a hollow housing; a button member installed in the housing; a needle member momentarily protruding to the outside of the housing from a standby position when the button member is pressed, and then returning to the standby position in the housing; and a UV LED installed in the housing and irradiating UV light onto the needle member at the standby position.

### CROSS REFERENCE TO RELATED APPLICATION

This patent document claims priority from and the benefits of Korean Patent Application No. 10-2014-0131981, filed on Sep. 30, 2014, entitled “UV LED INTEGRATED FLEAM”, the entirety of which is incorporated by reference.

### TECHNICAL FIELD

The present disclosure relates to a fleam or devices for bloodletting.

### BACKGROUND

In general, bloodletting or exsanguination is performed for medical examination or treatment of patients. When a large amount of blood is extracted, a syringe is used. However, when a small amount of blood is exsanguinated, for example, when the sugar content in blood of a diabetic patient or a blood type is examined, a fleam is used to perform exsanguination or bloodletting. In some medical practices, including the oriental medicine practices in Asia, when extravasated blood is taken up or a cupping treatment is applied, a fleam is used to prick the skin of a patient several or several tens of times.

### SUMMARY

Various embodiments are directed to a fleam which sterilizes a needle therein while the fleam is used, thereby preventing a user from being infected by the needle. In some implementations, a fleam can structured to include a feature of sterilizing a needle using a UV LED mounted in the fleam.

Also, various embodiments are directed to a fleam which includes a UV LED to irradiate a peak wavelength of UV light having an excellent sterilization effect onto a needle.

In an embodiment, a fleam may include: a hollow housing; a button member installed in the housing; a needle member momentarily protruding to the outside of the housing from a standby position when the button member is pressed, and then returning to the standby position in the housing; and a UV LED installed in the housing and irradiating UV light onto the needle member at the standby position.

The fleam may further include a reflecting plate installed around the inside of the housing having the UV LED installed therein.

The needle member may be moved together with an elevated body moved in the housing. At the position where the needle member protrudes to the outside of the housing, the elevated body may cover the UV LED.

In an embodiment, a fleam may include: a hollow housing; a button member installed in the housing; a needle member momentarily lowered from a standby position when the button member is pressed, and then returning to the standby position; a sterilization cover coupled to the bottom of the housing so as to be lifted and lowered; and a UV LED installed in the sterilization cover and irradiating UV light onto the needle member at the standby position.

The fleam may further include a reflecting plate installed around the inside of the sterilization cover having the UV LED installed therein.

At a position where the sterilization cover is lifted with respect to the housing, the needle member may protrude to the outside of the sterilization cover when the needle member is lowered. At a position where the sterilization cover is lowered with respect to the housing, the needle member may not protrude to the outside of the sterilization cover when the needle member is lowered.

At the position where the sterilization cover is lifted with respect to the housing, power may be supplied to the UV LED.

The needle member may be moved together with the elevated body moved in the housing. At a position where the needle member protrudes to the outside of the sterilization cover, the elevated body may cover the UV LED.

The needle member may be attached to or detached from the elevated body in the lateral direction of the elevated body, the housing may have a slit formed at a position to expose the attachment/detachment portion of the elevated body, and the sterilization cover may have a slit corresponding to the slit of the housing.

The sterilization cover may be rotated at a predetermined angle in the side-to-side direction with respect to the housing, and the rotation angle of the sterilization cover with respect to the housing may determine whether the slit of the housing coincides with the slit of the sterilization cover.

When the slit of the housing deviates from the slit of the sterilization cover, power may be supplied to the UV LED.

The housing may have two slits formed at positions facing each other, the sterilization cover may also have two slits formed at positions facing each other, the elevated body may have a fastening groove formed through both side surfaces and having the same cross-sectional surface at both side surfaces, and the needle member may include a fastening part having a shape corresponding to the fastening groove and a needle extended from the bottom of the fastening part.

The sterilization cover may be elastically supported in the direction that the sterilization cover is lowered with respect to the housing.

The housing may have a “[” shaped groove formed therein, the sterilization cover may have a protrusion formed thereon, the protrusion of the sterilization cover may be seated to the “[” shaped groove of the housing, and the sterilization cover may be moved by being guided along the “[” shaped structure with respect to the housing.

In an embodiment, a sterilization cover which sterilizes a needle member of a fleam may include: a coupling unit coupled to a housing of the fleam; a needle hole which is formed at an end thereof and through which a needle of the needle member passes; and a UV LED installed on the inner surface thereof.

The sterilization cover may further include a reflecting plate installed around the inside of the sterilization cover having the UV LED installed therein.

### BRIEF DESCRIPTION OF THE DRAWINGS

FIG. 1 is an exploded perspective view of a fleam in accordance with an embodiment of the present invention.

FIG. 2 is a front cross-sectional view illustrating the position of a sterilization cover with respect to a housing in a state where the fleam is stored or a needle is replaced.

FIG. 3 is a front cross-sectional view illustrating the position of the sterilization cover with respect to the housing in a state where the fleam in accordance with the embodiment of the present invention is being used.

FIG. 4 is a diagram illustrating the positional relation between the housing and the sterilization cover in accordance with the embodiment of the present invention.

### DETAILED DESCRIPTION

The disclosed technology can be used to sterilize a fleam via UV light and to address certain undesired issues in other fleam designs without such UV light sterilization.

For example, various conventional fleams for bloodletting are operated as follows. A needle of such a fleam is momentarily pushed into the skin through a cap housing and pulled out of the skin by a spring installed in a cylindrical body. Since the needle of the fleam comes in contact with blood, a replaceable and disposable needle can be used to minimize contamination or infection. With disposable needles used in fleams, users do not feel that the needle of the conventional fleam needs to be sterilized.

However, when operation is continuously performed on the same subject, the needle mounted on the main body of the fleam is not replaced during the operation. That is, when the fleam is used several or several tens of times for the same subject (for example, before a cupping treatment is applied), a time interval exists from the time at which a certain portion is hit to the time at which another portion is hit. Thus, the fleam is likely to be contaminated. When an operator is pricked by the needle during the operation, the operator may be infected through the blood of the subject.

That is, although a disposable needle is used in various fleams, the needle is likely to be contaminated by a user's hand or blood or contaminants in the air, while the skin of the subject is repetitively hit by the fleam. Thus, the needle may serve as a source of infection to the subject or operator. Furthermore, while a used needle is replaced, the operator may be pricked by the needle. Thus, although a disposable needle is used, the disposable needle needs to be sterilized. The disclosed technology can be used to mitigate this drawback in such fleams by providing UV light sterilization within the fleams.

The present disclosure may be embodied in different forms and should not be constructed as limited to the embodiments set forth herein. Rather, these embodiments are provided so that this disclosure will be thorough and complete, and will fully convey the scope of the disclosure to those skilled in the art. Throughout the disclosure, like reference numerals refer to like parts throughout the various figures and embodiments of the disclosure.

Furthermore, the structure or configuration of any one embodiment among the following embodiments can be applied to or substituted with the structure or configuration of another embodiment by those skilled in the art, if necessary. The configuration may be deleted or another configuration may be added.

Hereafter, various embodiments of the present invention will be described in detail with reference to the accompanying drawings.

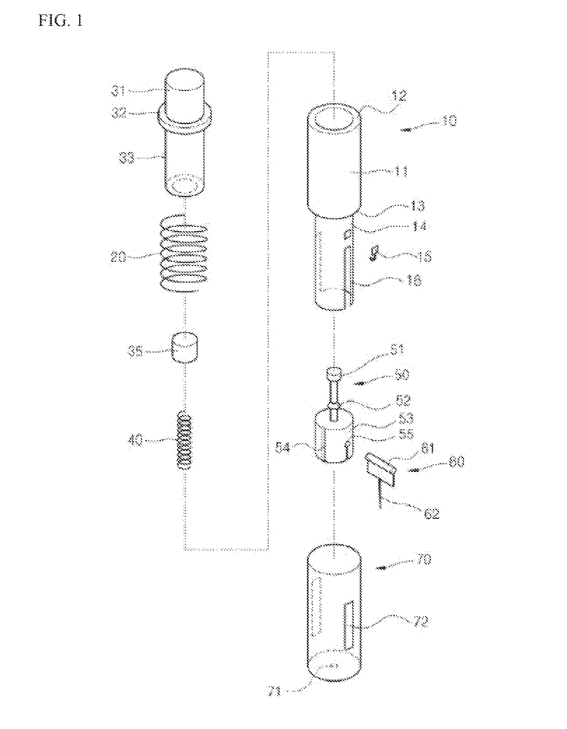

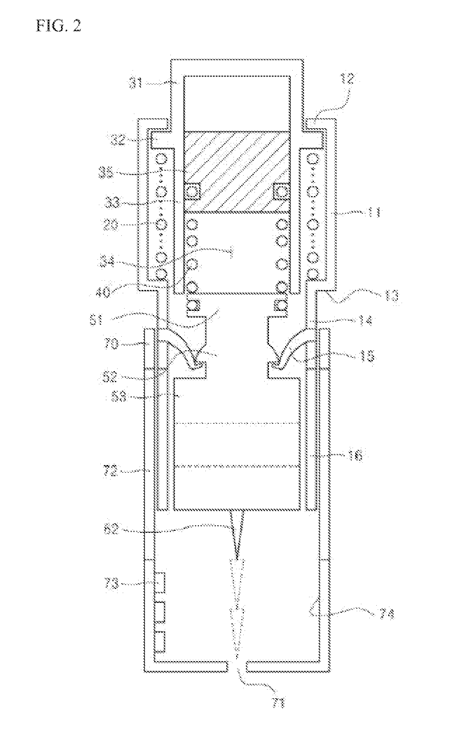

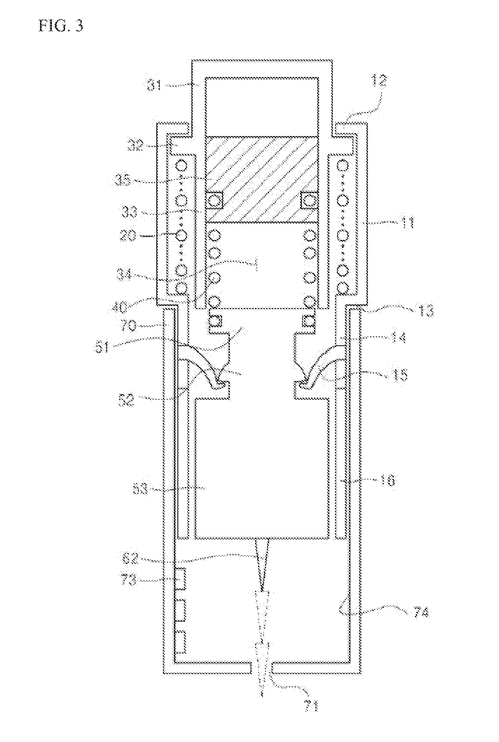

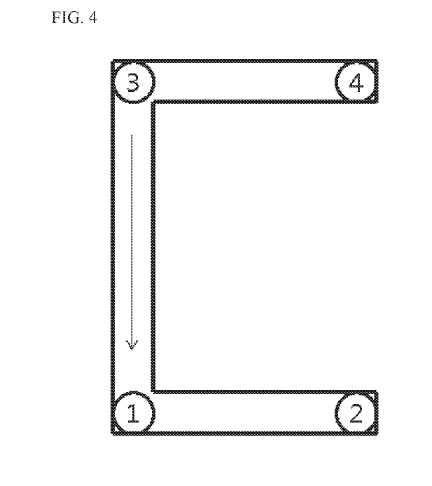

FIG. 1 is an exploded perspective view of a fleam in accordance with an embodiment of the present invention. FIG. 2 is a front cross-sectional view illustrating the position of a sterilization cover with respect to a housing in a state where the fleam is stored or a needle is replaced. FIG. 3 is a front cross-sectional view illustrating the position of the sterilization cover with respect to the housing in a state where the fleam in accordance with the embodiment of the present invention is being used. FIG. 4 is a diagram illustrating the positional relation between the housing and the sterilization cover in accordance with the embodiment of the present invention.

The fleam in accordance with the embodiment of the present invention includes a housing 10, a button member 30, an elevated body 50, a needle member 60, and a sterilization cover 70. The housing 10 serves as a body such as the body of a pen. The button member 30 is installed to function as a button such as a button of a pen. The elevated body 50 and the needle member 60 are installed to function as a needle such as a pen lead. The sterilization cover 70 is installed to function as a cover such as a lid of a pen.

The above components will be described in detail as follows. First, the housing 10 includes a large-diameter part 11, an upper jaw 12 formed at the top of the large-diameter part 11, a lower jaw 13 formed at the bottom of the large-diameter part 11, a small-diameter part 14 formed at the bottom of the lower jaw 13, a slit 16 formed at the side surface of the small-diameter part 14, and a fixed spring 15 formed on the inner surface of the small-diameter part 14 so as to protrude inward.

The button member 30 has a hollow part 34 opened downward, and includes a button part 31, a diameter-expanded part 32, a spring insertion part 33, and a second spring top fixing part 35. The diameter-expanded part 32 is formed at the bottom of the button part 31. The spring insertion part 33 is extended from the bottom of the diameter-expanded part 32. The second spring top fixing part 35 is inserted into and fixed to the hollow part 34.

Referring to FIGS. 2 and 3, the coupling relation between the housing 10 and the button member 30 will be described as follow. The diameter-expanded part 32 having a slightly smaller diameter than the inner diameter of the large-diameter part 11 is inserted into the large-diameter part 11. The diameter-expanded part 32 is stopped by the upper jaw 12 in the upward direction, and stopped by the lower jaw 13 in the downward direction. Thus, the button member 30 can be moved with respect to the housing 10 by the distance at which the diameter-expanded part 32 is moved upward/downward between the upper jaw 12 and the lower jaw 13.

The spring insertion part 33 extended from the bottom of the diameter-expanded part 32 has a smaller outer diameter than the small-diameter part 14. Thus, the spring insertion part 33 can be inserted into the small-diameter part 14 so as to be moved upward/downward. Between the outer circumference of the spring insertion part 33 and the inner circumference of the large-diameter part 11, a first spring 20 is inserted. The bottom of the first spring 20 is supported by the top surface of the lower jaw 13, and the top of the first spring 20 is supported by the bottom surface of the diameter-expanded part 32. Thus, the first spring 20 elastically supports the button member 30 upward with respect to the housing. Therefore, when the button part 31 of the button member 30 protruding upward from the upper jaw 12 is pressed, the first spring 20 resists the pressing force. Then, as soon as the pressing force applied to the button part 31 is released, the button member 30 returns to the position illustrated in FIGS. 2 and 3.

The elevated body 50 has the second spring bottom fixing part 51 formed at the top thereof, a fixed jaw 52 formed at a position which is slightly separated from the bottom of the second spring bottom fixing part 51, and a body part 53 formed at the bottom of the fixed jaw 52. The body part 53 has a guide part 54 and a fastening groove 55. The guide part 54 is formed on the side surface of the body part 53 so as to extend in the vertical direction, and the fastening groove 55 is formed through the side surface of the body part 53.

The needle member 60 includes a fastening part 61 and a needle 62 and is inserted into the fastening groove 55 in the lateral direction. The fastening part 61 has the same cross-sectional shape as the shape of the fastening groove 55, and the needle 62 is fixed to the bottom center of the fastening part 61. The fastening groove 55 is formed in such a shape that the needle member 60 is reliably fixed in the vertical direction with respect to the elevated body 50, and slid in the side-to-side direction. The needle member 60 and the elevated body 50 may be stiffly coupled to each other. In this case, when a force is not applied, the needle member 60 may not be moved in the side-to-side direction with respect to the elevated body 50. Only when a force is applied, the needle member 60 may be pushed in the side-to-side direction.

Referring to FIGS. 2 and 3, the coupling relation between the elevated body and the needle member and the housing will be described as follow. The elevated body and the needle member are moved together within the housing. Since the outer diameter of the body part 53 is slightly smaller than the inner diameter of the small-diameter part 14, the body part 53 can be moved upward/downward with respect to the small-diameter part 14. At this time, since a guide part (not illustrated) formed on the inner surface of the small-diameter part 14 is engaged with the guide part 54 of the body part 53, the elevated body 50 is prevented from rotating in the side-to-side direction, even though the elevated body 50 can be moved upward/downward with respect to the small-diameter part 14. In such a state where the elevated body 50 is prevented from rotating in the side-to-side direction, both side ends of the fastening groove 55 of the body part 53 are exposed through the respective slits 16. The fixed jaw 52 is locked to the top of the fixed spring 15 protruding to the inside of the housing. The fixed spring 15 is elastically supported in the direction to lift the fixed jaw 52.

The second spring 40 is inserted into the hollow part 34 of the button member 30. The bottom of the second spring 40 is fixed to the second spring bottom fixing part 51, and the top of the second spring 40 is fixed to the second spring top fixing part 35 fixed to the hollow part of the button member 30. The bottom of the second spring 40 is completely fixed to the second spring bottom fixing part 51 in the vertical direction, and the top of the second spring 40 is completely fixed to the second spring top fixing part 35 in the vertical direction. In the state of FIGS. 2 and 3, the second spring 40 is compressed more slightly than the state that the second spring 40 is neither tensioned nor compressed.

Hereafter, the operation of the fleam will be described briefly. As an operator presses the button part 31, the button member 30 and the second spring top fixing part 35 are lowered in a state where the fixed jaw 52 is locked to the fixed spring 15. Thus, the second spring 40 starts to be gradually compressed between the second spring top fixing part 35 and the second spring bottom fixing part 51.

When the operator further presses the button part 31 such that the bottom of the spring insertion part 33 presses the fixed spring 15, the fixed spring 15 is spread to release the fixed jaw 52 which has been locked to the fixed spring 15. As a result, the second spring 40 which has been compressed while the button member 30 is getting lowered begins to be elongated by the elastic force thereof. Then, the elevated body 50 is quickly lowered.

As the second spring 40 is elongated, the length of the second spring 40 is further increased by the elastic force of the second spring 40 (first state), compared to the state in which the second spring 40 is neither tensioned nor compressed. Then, the length of the second spring 40 is reduced in a moment and set to the length in the state in which the second spring 40 is neither tensioned nor compressed (second state). Thus, the needle member 60 and the elevated body 50 fixed to the bottom of the second spring 40 are also momentarily set in the first state in which the needle comes out to the lowermost position (refer to the lowermost dotted triangle of FIGS. 2 and 3) and then immediately set in the second state in which the needle is slightly lifted (refer to the dotted triangle over the lowermost dotted triangle of FIGS. 2 and 3).

At this time, the first state corresponds to a state in which the operator pricks the skin of a subject using the needle, and the second state corresponds to a state in which the needle is pulled out of the skin of the subject.

When the operator releases the button part 31, the first spring 20 which has been compressed pushes the diameter-expanded part 32 such that the button member 30 and the second spring top fixing part 35 moved with the button member 30 are lifted. Thus, as the second spring 40 is momentarily tensioned, the length of the second spring 40 is increased more than in the state where the second spring 40 is neither tensioned nor compressed. Therefore, the length of the second spring 40 rapidly returns to the original length, due to the elastic restoring force of the second spring 40. As a result, the elevated body 50 fixed to the bottom of the second spring 40 is also lifted. The upper part of the fixed jaw 52 of the elevated body 50 forms an inclined surface. Thus, when the fixed jaw 52 is lifted, the fixed spring is spread while the bottom surface of the fixed spring 15 comes in contact with the upper inclined surface of the fixed jaw 52. Then, the fixed jaw 52 is lifted over the fixed spring 15 again. FIGS. 2 and 3 illustrate a state in which the distance between the fixed position of the needle 62 and the lowermost position of the needle 62 and the distance between the second spring bottom fixing part 51 and the fixed jaw 52 is exaggerated. However, when the lengths thereof are properly determined during a manufacturing process, the above-described operation can be performed without any problems.

Then, the sterilization cover 70 will be described. The sterilization cover 70 is opened upward, and has a needle hole formed in the bottom center thereof. Furthermore, the sterilization cover 70 has slits formed at both sides thereof. The sterilization cover 70 has a UV LED 73 installed at the bottom of the inner circumference thereof and a reflecting plate 74 installed on the inner circumference thereof. The reflecting plate 74 can reflect UV light.

Since the inner diameter of the sterilization cover 70 is slightly larger than the outer diameter of the small-diameter part 14 of the housing, the sterilization cover 70 can be moved upward/downward with respect to the small-diameter part of the housing. The sterilization cover 70 is installed in the housing so as to be moved upward/downward only at a section between the position of FIG. 2 and the position of FIG. 3. When the sterilization cover is lowered, the needle 62 does not protrude to the outside of the needle hole 71 even though the needle 62 is lowered to the lowermost position (first state). Thus, although the button part is pressed by mistake, the subject can be prevented from being pricked by the needle 62.

In a state where the sterilization cover is lifted, the needle 62 protrudes to the outside of the needle hole 71 when the needle 62 is lowered to the lowermost position (first state), or is positioned in the needle hole 71 when the needle 62 is slightly lifted (second state). Thus, when the sterilization cover is lifted as illustrated in FIG. 3, the fleam can be used as in a typical case.

At a part where the sterilization cover and the small-diameter part face each other, a protrusion which is illustrated in FIG. 4 by circles is formed on the sterilization cover, and a “[” shaped groove which is illustrated in FIG. 4 as a channel for the circles is formed in the small-diameter part. Then, when the protrusion is inserted into the groove, the sterilization cover is not rotated in case where the sterilization cover is lifted upward or downward with respect to the small-diameter part (between positions 1 and 3 of FIG. 4), and can be rotated left and right at a predetermined angle with respect to the small-diameter part, in case where the sterilization cover is lifted to the uppermost position (between positions 3 and 4 of FIG. 4) or lowered to the lowermost position (between positions 1 and 2 of FIG. 4). As such, when the sterilization cover is rotated left and right at a predetermined angle with respect to the small-diameter part in a state where the sterilization cover is lifted to the uppermost position or lowered in the lowermost position (position 4 or 2 of FIG. 4), the sterilization cover can be prevented from moving upward or downward with respect to the small-diameter part. At this time, when the structure which elastically supports the sterilization cover 70 to descend with respect to the housing is applied (refer to the arrow direction of FIG. 4), the sterilization cover 70 maintains a state where the sterilization cover 70 is lowered with respect to the housing at all times.

At the position where the sterilization cover can be lifted or lowered with respect to the small-diameter part (position 1 or 3 of FIG. 4), the slit 16 of the small-diameter part and the slit 72 of the sterilization cover coincide with each other (refer to FIG. 2). At the position where the sterilization cover is rotated at a predetermined angle so as not to be lifted or lowered with respect to the small-diameter part (position 2 or 4 of FIG. 4), the slit 16 of the small-diameter part and the slit 72 of the sterilization cover deviate from each other (refer to FIG. 3).

Thus, the needle member 60 can be replaced at the position 1 of FIG. 4 (refer to FIG. 2). That is, when a new needle member is pushed into one side of the coincided slits in a state where the used needle member is inserted into the elevated body, the used needle member is pushed out of the opposite coincided slits by the new needle member.

In the state where the slits coincide with each other as illustrated in FIG. 2, foreign matters such as dust can be introduced into the sterilization cover through the slits. Thus, when the fleam is stored in the state in which the sterilization cover is rotated in one direction from the state of FIG. 2 such that the slits deviate from each other (position 2 of FIG. 4), the needle 62 is not exposed to the outside even though the button part 31 is pressed by mistake. Furthermore, foreign matters such as dust are not introduced into the sterilization cover.

When the operator is intended to use the fleam, the operator rotates the sterilization cover from the position 2 to the position 1 of FIG. 4, lifts the sterilization cover to the position 3 while overcoming the elastic force, and rotates the sterilization cover to the position 4. Then, the fleam is set in the state illustrated in FIG. 3. In this state, since the slits deviate from each other, the slits are blocked. Furthermore, when the operator presses the button part 31, the needle 62 can protrude the outside of the needle hole 71 (that is, available state).

As described above, the sterilization cover 70 has the UV LED 73 installed at the bottom of the inner circumference thereof and a reflecting plate 74 installed on the inner circumference thereof. In this state, a battery (not illustrated) for supplying power to the UV LED 73 may be mounted in the sterilization cover 70, and power may be supplied to the UV LED 73 only when the sterilization cover is at the position 4 of FIG. 4. Then, UV sterilization is performed only while the fleam is used. That is, at this position, UV light irradiated from the UV LED sterilizes the needle 62 while being reflected by the reflecting plate 74.

In this state, when the button part 31 is pressed, the needle 62 is lowered, and the body part 53 lowered with the needle 62 covers the UV LED 73. Thus, the UV light irradiated from the UV LED is blocked by the body part at the moment the needle is lowered. At the point of time that the needle is lowered to block the UV light, the needle almost meets the skin of the subject. In this aspect, the structure in accordance with the embodiment of the present invention can have an effect of blocking even a small amount of UV leaking through the needle hole 71, when the fleam is close to the skin of the subject.

In accordance with the embodiments of the present invention, when the fleam is used, UV light may be continuously irradiated onto the needle. Thus, the operator handling the fleam can be prevented from getting infected by the needle.

While various embodiments have been described above, it will be understood to those skilled in the art that the embodiments described are by way of example only. Accordingly, the disclosure described herein should not be limited based on the described embodiments.

What is claimed is:

1. A fleam comprising:
a hollow housing;
a button member installed in the housing;
a needle member momentarily protruding to the outside of the housing from a standby position when the button member is pressed, and then returning to the standby position in the housing; and
a UV LED installed in the housing and irradiating UV light onto the needle member at the standby position.

2. The fleam of claim 1, further comprising a reflecting plate installed around the inside of the housing having the UV LED installed therein.

3. The fleam of claim 1, wherein the needle member is moved together with an elevated body moved in the housing, and
at the position where the needle member protrudes to the outside of the housing, the elevated body covers the UV LED.

4. A fleam comprising:
a hollow housing;
a button member installed in the housing;
a needle member momentarily lowered from a standby position when the button member is pressed, and then returning to the standby position;
a sterilization cover coupled to the bottom of the housing so as to be lifted and lowered; and
a UV LED installed in the sterilization cover and irradiating UV light onto the needle member at the standby position.

5. The fleam of claim 4, further comprising a reflecting plate installed around the inside of the sterilization cover having the UV LED installed therein.

6. The fleam of claim 4, wherein at a position where the sterilization cover is lifted with respect to the housing, the needle member protrudes to the outside of the sterilization cover when the needle member is lowered, and
at a position where the sterilization cover is lowered with respect to the housing, the needle member does not protrude to the outside of the sterilization cover when the needle member is lowered.

7. The fleam of claim 6, wherein at the position where the sterilization cover is lifted with respect to the housing, power is supplied to the UV LED.

8. The fleam of claim 4, wherein the needle member is moved together with the elevated body moved in the housing, and
at a position where the needle member protrudes to the outside of the sterilization cover, the elevated body covers the UV LED.

9. The fleam of claim 4, wherein the needle member is attached to or detached from the elevated body in the lateral direction of the elevated body,
the housing has a slit formed at a position to expose the attachment/detachment portion of the elevated body, and the sterilization cover has a slit corresponding to the slit of the housing.

10. The fleam of claim 9, wherein the sterilization cover is rotated at a predetermined angle in the side-to-side direction with respect to the housing, and the rotation angle of the sterilization cover with respect to the housing determines whether the slit of the housing coincides with the slit of the sterilization cover.

11. The fleam of claim 10, wherein when the slit of the housing deviates from the slit of the sterilization cover, power is supplied to the UV LED.

12. The fleam of claim 9, wherein the housing has two slits formed at positions facing each other,
the sterilization cover also has two slits formed at positions facing each other,
the elevated body has a fastening groove formed through both side surfaces and having the same cross-sectional surface at both side surfaces, and
the needle member comprises a fastening part having a shape corresponding to the fastening groove and a needle extended from the bottom of the fastening part.

13. The fleam of claim 4, wherein the sterilization cover is elastically supported in the direction that the sterilization cover is lowered with respect to the housing.

14. The fleam of claim 10, wherein the housing has a “[” shaped groove formed therein, the sterilization cover has a protrusion formed thereon, the protrusion of the sterilization cover is seated to the “[” shaped groove of the housing, and the sterilization cover is moved by being guided along the “[” shaped structure with respect to the housing.

15. A sterilization cover which sterilizes a needle member of a fleam, comprising:
a coupling unit coupled to a housing of the fleam;
a needle hole which is formed at an end thereof and through which a needle of the needle member passes; and
a UV LED installed on the inner surface thereof.

16. The sterilization cover of claim 15, further comprising a reflecting plate installed around the inside of the sterilization cover having the UV LED installed therein.
